(19) United States
(12) Patent Application Publication
(10) Pub. No.: US 2001/0013345 A1
(43) Pub. Date: Aug. 16, 2001

(54) Transpharyngeal tube

(76) Inventor: Volker Bertram, Sulz (DE)

Correspondence Address: Eugene LeDonne, Esq., Reed Smith LLP, 17th Floor, 375 Park Ave, New York, NY, 10152, US

(21) Appl. No.: 09/745,271

(22) Filed: Dec. 21, 2000

(30) Foreign Application Priority Data
Dec. 23, 1999 (DE) DE 199 62 372.4

(51) Int. Cl. A61M 16/00; A62B 9/06

(52) U.S. Cl. 128/200.26; 128/207.14; 128/207.15

(57) ABSTRACT

A transpharyngeal tube for intubation anaesthesia which includes a tube shaft with an inflatable cuff for blocking the pharynx and an inflatable esophageal cuff of the tube shaft for blocking the esophagus. A common ventilation channel for ventilating and deaerating the two cuffs is provided within or on the shaft wall. Design and handling of the transpharyngeal tube have been facilitated.

### RELATED PATENTS AND APPLICATIONS

This application claims Paris Convention priority of DE 199 62 372.4 filed on Dec. 23, 1999, the complete disclosure of which is hereby incorporated by reference.

### FIELD OF INVENTION

The invention relates to a transpharyngeal tube and, more particularly, to a transpharyngeal tube for intubation anaesthesia which includes a tube shaft with an inflatable pharyngeal cuff for blocking the pharynx and an inflatable esophageal cuff for blocking the esophagus.

### BACKGROUND OF THE INVENTION

A transpharyngeal tube of this type is disclosed e.g. in DE 195 37 735 C1.

A transpharyngeal tube is an alternative to artificial respiration by means of a so-called larynx mask or also to endotracheal tubes if there is no danger of aspiration. Due to its simplicity, the transpharyngeal tube is an ideal supplement for emergency artificial respiration.

A possible application of intubation anaesthesia includes, in most cases after premedication of the patient, initial intravenous anaesthesia or after inhalation anaesthesia, introduction of the transpharyngeal tube into the patient. If the tip of the tube is introduced only to the entrance region of the esophagus, and the esophageal cuff blocks only said entrance region, painful strain of the esophagus is prevented. The activated esophageal cuff occludes the entrance to the esophagus in a gas and liquid-tight manner.

When the transpharyngeal tube has been introduced, the trachea is sealed off by blocking the cuff in the esophagus and in the pharynx to provide at any time a free respiratory path for the patient, separate from the esophagus, by means of an application channel of the tube, and to prevent in particular any mixing of vomitted stomach contents and respiratory air. Artificial respiration of the patient is now possible via the application channel in the inside of the transpharyngeal tube. It is possible to add an inhalation anaesthesia by supplying anaesthetic, e.g. an air or O₂ anaesthetic mixture.

The application of the transpharyngeal tube requires at first inflation of the pharyngeal cuff to position and stabilize the transpharyngeal tube. The known transpharyngeal tube comprises two separate ventilation lines for each cuff which requires use of two pressure manometers.

It is the underlying purpose of the present invention to facilitate construction and handling of the transpharyngeal tube.

### SUMMARY OF THE INVENTION

The above-mentioned object is achieved by a transpharyngeal tube, in particular for intubation anaesthesia, which is provided within or on the shaft wall of the tube with a common ventilation channel for ventilation and deaeration of the pharyngeal cuff and also the esophageal cuff. Two cuffs can be inflated in a controlled and adjustable manner via one single line or one single channel.

In a preferred embodiment, the ventilation channel between the pharyngeal cuff and the esophageal cuff comprises a channel section having a flow cross-section of reduced size with respect to the flow cross-section of the channel section leading to the pharyngeal cuff. These flow-geometric proportions have the consequence that first of all, the larger pharyngeal cuff is always ventilated, inflated and stabilized. Subsequently, the air flows via the channel section of reduced cross-section, into the esophagus cuff, ventilates same and at the same time automatically adopts the same pressure as within the pharyngeal cuff. This essentially facilitates handling of the inventive transpharyngeal tube.

If the pharyngeal cuff and/or the esophageal cuff are made from an elastic material, such as rubber or silicone, the pressure compensation between the two cuffs is supported since the cuffs are resilient and easy to deform. Likewise, pressure impact through inner walls of the hollow organ into which the transpharyngeal tube has been introduced, can be compensated for by the communicating cuffs.

The pharyngeal cuff and/or the esophageal cuff may be designed as low-pressure cuffs and pressure cuffs. Low-pressure cuffs have the advantage that the tissue surfaces of the pharynx or the esophagus entrance where the cuffs abut, are protected.

Preferably, the esophageal cuff surrounds a tube tip of the transpharyngeal tube on all sides. This increases protection of the patient during introduction of the tube tip into the esophagus entrance and positioned therein.

Both the pharyngeal cuff and the esophageal cuff may have large volumes and adapt well to the individual anatomic situation.

One embodiment of the inventive transpharyngeal tube is shown in the schematic drawing and is explained in the following description with reference to the drawing.

### BRIEF DESCRIPTION OF THE DRAWING

FIG. 1 shows the entrance region of the esophagus into which a transpharyngeal tube has been introduced;

FIG. 2 shows an enlarged partial region of the transpharyngeal tube in accordance with FIG. 1.

### DESCRIPTION OF THE PREFERRED EMBODIMENT

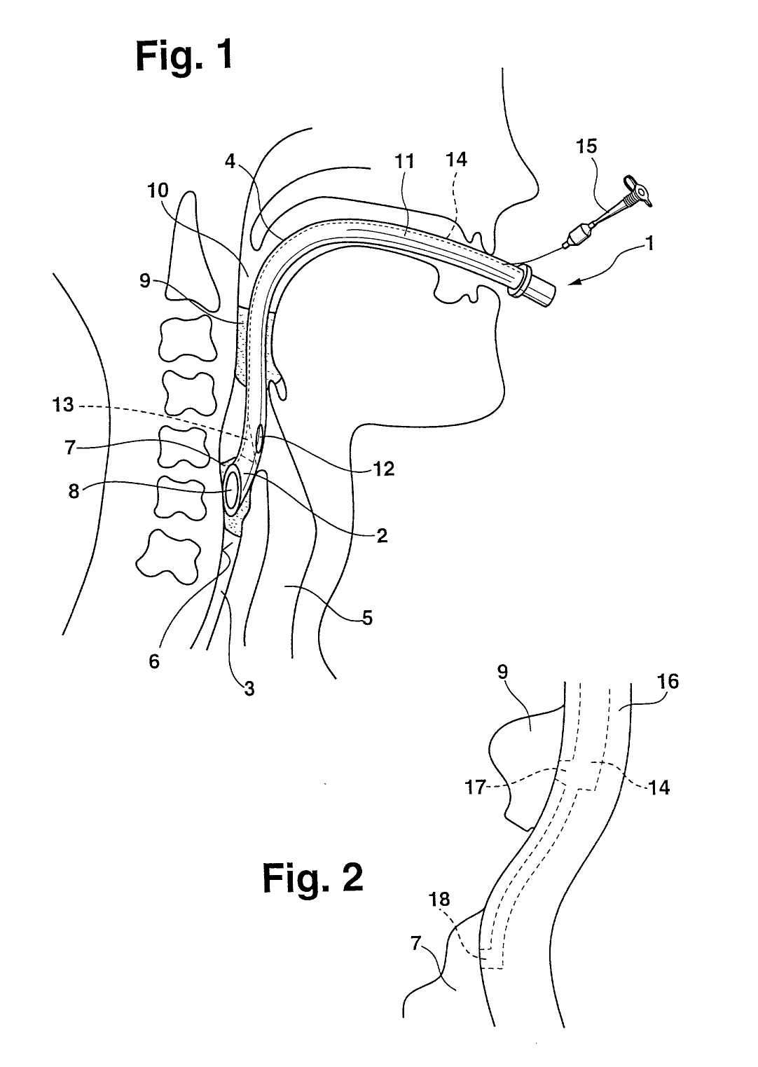

FIG. 1 shows the application of a transpharyngeal tube 1 whose tube tip 2 has been introduced into the entrance region of the esophagus 3. The transpharyngeal tube 1 comprises a tube shaft 4 having an S-shaped longitudinal profile or being straightened at least along the length of the two cuffs. The longitudinal profile formed in this fashion, guarantees that the transpharyngeal tube 1 is always introduced into the entrance region of the esophagus 3 and not wrongly into the trachea 5. Due to the S-shape of the tube shaft 4 made from a flexible elastic plastic material, the tube tip 2 rests with slight pressure on the inner wall 6 of the esophagus 3 due to the spring effect of the tube shaft 4 thereby fixing the tube tip 2 in the entrance region of the esophagus 3. To maintain the tube tip 2 in this position, an esophageal cuff 7 is ventilated blocking the entrance region of the esophagus 3 in a gas and liquid-tight manner. When the transpharyngeal tube 1 is introduced, the esophageal cuff 7 is folded and put into a cavity 8 of the tube tip 2 to ensure easy insertion of the transpharyngeal tube 1. The esophageal cuff 7 may be disposed at the tube tip 2 in such a fashion that it does not increase the outer diameter of the tube tip 2.

The pharynx 10 is proximally blocked via an inflatable pharyngeal cuff 9. The esophagus entrance is distally blocked by the esophageal cuff 7. This produces and end-to-end connection between the trachea (larynx) 5 as natural respiratory path and the transpharyngeal tube 1 via an application channel 11 without reducing the lumen of the transpharyngeal tube 1 with respect to the trachea 5. On the one hand, the treated patient is artificially supplied with air via the application channel 11 and on the other hand anaesthesized by supplied application means, such as anaesthesia.

Application means may flow into the trachea 5 via the application channel 11 and an application opening 12. The application channel 11 is continuously formed in the tube shaft 4 and is subdivided by a blocking element 13 into an upper part used for application and the cavity of the tube tip 2. The blocking element 13 forms a flow aid which may guide inflowing application means, a catheter or fiber bronchoscope to the application opening 12.

The pharyngeal cuff 9 and the esophageal cuff 7 may be ventilated and inflated or deaerated in a controlled manner with adjustable inner pressure via a common ventilation channel 14 formed in the tube shaft 4 and extending in the wall of the tube shaft 4. The ventilation channel 14 extends from a blocking device 15 for opening and closing the ventilation channel 14 to the esophageal cuff 7. It is possible to connect known means for inflating a cuff and common pressure manometers to the blocking device 15. The ventilation channel 14 has a ventilation opening within the pharyngeal cuff 9 and a ‘ventilation opening within the esophageal cuff 7 thereby interconnecting the two cuffs 7 and 9 via the ventilation channel 14 to allow pressure compensation between the two cuffs 7 and 9. Pressure acting on the esophageal cuff 7 may be decreased e.g. via the larger pharyngeal cuff 9.

FIG. 2 shows the cooperation of the two cuffs 7 and 9. The ventilation channel 14 within the shaft wall 16 has a larger flow cross-section between the blocking device 15 (see FIG. 1) and the ventilation opening 17 within the pharyngeal cuff 9 for ventilation.

The flow cross-section of the ventilation channel 14 between the ventilation opening 17 and the ventilation opening 18 within the esophageal cuff 7 is reduced with the effect that during simultaneous ventilation of the cuffs 7 and 9, the pharyngeal cuff 9 is automatically inflated at first. Due to the flow proportions, the pharyngeal cuff 9 is automatically deaerated first during simultaneous deaeration of both cuffs 7 and 9. If an inflated cuff 7 or 9 is additionally pressurized via the inner wall 6 of the hollow organ (see FIG. 1), this pressure can be decreased via the other cuff 7 or 9 since both cuffs may communicate with one another via the common ventilation channel 14. Air exchange between the cuffs 7 and 9 is possible at any time.

I claim:

1. A transpharyngeal tube used for intubation anaesthesia, said tube comprising:
a tube shaft with an inflatable pharyngeal cuff for blocking the pharynx, and
an inflatable esophageal cuff for blocking the esophagus,
wherein, within or on the shaft wall of the tube shaft, a common ventilation channel for ventilating and deaerating the two cuffs is provided.

2. The transpharyngeal tube according to claim 1, wherein the tube shaft further comprises at least one of an S-shaped longitudinal profile and a straightened stretch at the level of the cuffs.

3. The transpharyngeal tube according to claim 1, wherein the ventilation channel further comprises, between the pharyngeal cuff and the esophageal cuff, a channel section having a flow cross-section which is reduced with respect to the flow cross-section of the channel section leading to the pharyngeal cuff.

4. The transpharyngeal tube according to claim 1, wherein at least one of the pharyngeal cuff and the esophageal cuff are made from an elastic material, such as rubber and silicone, or of other elastomers, such as PVC and polyethylene.

5. The transpharyngeal tube according to claim 1, wherein at least one of the pharyngeal cuff and the esophageal cuff are formed as low-pressure cuffs.

6. The transpharyngeal tube according to claim 1, wherein the esophageal cuff surrounds a tube tip of the transpharyngeal tube on all sides.
